(19) United States
(12) Patent Application Publication
(10) Pub. No.: US 2019/0292512 A1
(43) Pub. Date: Sep. 26, 2019

(54) ALGAE TREATMENT METHOD

(71) Applicant: Kao Corporation, Tokyo (JP)

(72) Inventors: Yoshitaka KUBOTA, Kawasaki-shi, Kanagawa (JP); Takafumi UEMATSU, Wakayama-shi, Wakayama (JP)

(73) Assignee: Kao Corporation, Tokyo (JP)

(21) Appl. No.: 16/302,294

(22) PCT Filed: May 2, 2017
(86) PCT No.: PCT/JP2017/017271

§ 371 Date: Nov. 16, 2018

(30) Foreign Application Priority Data
May 24, 2016 (JP) 2016-103384
Dec. 8, 2016 (JP) 2016-238776

(51) Int. Cl. C12N 1/12; C12P 7/64; C07C 29/149; C07C 67/02; C07C 51/09

(52) U.S. Cl. CPC C12N 1/12; C12P 7/64; C07C 51/09; C07C 67/02; C07C 29/149

(57) ABSTRACT

An algae treatment method includes: a first step of culturing microalgae in a culture medium having an initial nitrogen concentration of less than 12 mg/L for three days or more; and a second step of performing a thermal treatment of the microalgae that has gone through the first step at a pH of 2.0 or more and 10.0 or less and a temperature of 35° C. or more and 80° C. or less.

### TECHNICAL FIELD

The present disclosure relates to an algae treatment method.

### BACKGROUND ART

Attention has been paid to a plant biomass technology of producing various components from minute plants such as algae. For example, studies have been made to produce lipids from algae so that the lipids can be used in various applications. Among others, the lipids from algae have been expected as a clean, next-generation energy source because the lipids, when used as a fuel, do not increase the emission of carbon dioxide, and do not compete with production of foods, unlike grain-based alcohols.

For industrial lipid production from the algae, it is important to increase the accumulation of lipids in algae, and efficiently recover the lipids accumulated.

As a method for increasing the accumulation of lipids in the algae, application of an environmental stress to the algae being cultured has been studied. For example, an attempt has been made to increase the accumulation of lipids by leaving the algae in a state of nitrogen deficiency (see, e.g., Patent Document 1).

Further, it has also been studied a method for recovery of lipids from the algae. In this method, the lipids are recovered from the algae whose cell wall has been destroyed or weakened through a treatment under a high pressure condition or a strong alkali or strong acid condition (see, e.g., Patent Document 2).

### CITATION LIST

### Patent Document

Patent Document 1: Japanese Unexamined Patent Publication No. 2015-142575
Patent Document 2: Japanese Unexamined Patent Publication No. 2012-520076

### SUMMARY

### Technical Problem

However, for producing lipids from algae on a commercial basis, it is required to recover lipids from algae under a condition as gentle and mild as possible. Treatments under the high pressure condition or the strong alkali or strong acid condition that have been studied before can increase the recovery ratio of lipids. However, it is difficult to commercially employ such treatments in view of energy applied and/or facilities required. The inventors of the present application have found another problem, i.e., the application of an environmental stress for the purpose of increasing the accumulation of lipids in the algae lowers the recovery ratio of lipid as compared with the case where no environmental stress is applied. In order to efficiently recover the lipid from the algae to which the environmental stress has been applied to increase the accumulation of lipid, a treatment method suitable for this situation is required.

The present disclosure is directed to provide a suitable method for treating the algae to which the environmental stress has been applied. Also disclosed herein is a method for producing lipid through recovery of the lipid from the algae thus treated. Further disclosed herein is a method for producing aliphatic alcohol and/or glycerin from fats and oils contained in the lipid thus produced.

### Solution to the Problem

According to an aspect of the present disclosure, an algae treatment method includes: a first step of culturing microalgae belonging to Heterokontophyta in a culture medium having an initial nitrogen concentration of less than 12 mg/L for three days or more; and a second step of performing a thermal treatment of the microalgae that have gone through the first step at a pH of 2.0 or more and 10.0 or less and a temperature of 35° C. or more and 80° C. or less.

### Advantages of the Invention

According to the algae treatment method of the present disclosure, even if the accumulation of lipid in the algae is increased through the application of an environmental stress, the recovery of the lipid from the algae can be as easy as, or easier than, the recovery from the algae with no environmental stress. Further, lipid can be produced from the algae that have been treated through the above-described method, and aliphatic alcohol and/or glycerin can be produced by the lipid thus produced.

### DESCRIPTION OF EMBODIMENTS

According to the present disclosure, the “microalgae belonging to Heterokontophyta” are microalgae belonging to the Division Heterokontophyta. The “microalgae” designate organisms which produce oxygen through photosynthesis except for Bryophyta, Pteridophyta, and Spermatophyta and have a cell size of 1-100 μm in diameter. The cell size is a longitudinal diameter of a cell measured with an optical microscope at 400-fold magnification. Examples of the microalgae belonging to Heterokontophyta may include microalgae of the classes Bacillariophyceae and Eustigmatophyceae. Examples of the microalgae of the class Bacillariophyceae may include microalgae of the genera Chaetoceros, Nitzschia, and Skeletonema. Examples of the microalgae of the class Eustigmatophyceae may include microalgae of the genus Nannochloropsis. Among these microalgae, the microalgae of the class Eustigmatophyceae are suitable, and of the genus Nannochloropsis are more suitable in view of lipid productivity and lipid recovery. Examples of the algae of the genus Nannochloropsis may include Nannochloropsis oculata, Nannochloropsis salina, and Nannochloropsis gaditana. The microalgae may be harvested from natural fields such as the sea, cultivated, or obtained commercially.

An algae treatment method according to this embodiment includes a first step of culturing the microalgae belonging to Heterokontophyta in a culture medium having an initial nitrogen concentration of less than 12 mg/L for three days or more, thereby applying an environmental stress to the microalgae, and a second step of performing a thermal treatment of the microalgae that has received the environmental stress at a pH of 2.0 or more and 10.0 or less and a temperature of 35° C. or more and 80° C. or less.

The inventors of the present application have compared the amount of lipid recovered from the microalgae that have gone through an alkali treatment and solvent extraction with the amount of lipid recovered from the microalgae that have gone through the solvent extraction only without the alkali treatment. The comparison has revealed the followings. Specifically, the amount of lipid recovery from the microalgae that have received no environmental stress does not greatly vary even if the microalgae are treated with alkali or not. In contrast, the amount of lipid recovery from the microalgae that have received the environmental stress becomes greater than the amount of lipid recovery from the microalgae with no environmental stress if the alkali treatment is done, but is just equivalent to it if no alkali treatment is done.

This indicates that the application of the environmental stress to the microalgae can increase the lipid accumulation in the algae, but makes the recovery of the lipid difficult as compared with the case where no environmental stress is applied to the microalgae. Even if the lipid accumulation in the algae is increased through the application of the environmental stress, its commercial value would greatly decrease if the recovery of the lipid requires a high pressure condition or a strong acid or strong alkali condition.

The inventors of the present application have found that if the algae, to which the environmental stress has been applied to increase the accumulation of lipid, are thermally treated at a pH of 2.0 or more and 10.0 or less and a temperature of 35° C. or more and 80° C. or less, the recovery of lipid from the algae can be as easy as, or easier than, that from the algae that have received no environmental stress.

In this context, the “environmental stress” is a stress that could be applied to the microalgae when they are left in a condition which is not suitable for their growth. For example, such a condition may include a situation where nourishment such as nitrogen falls below a level optimum for the growth of the microalgae, or a situation where the concentration of salt, temperature, or density of growth, of the microalgae are different from those optimum for their growth.

### <Culturing Step>

In this embodiment, prior to the first step of applying the environmental stress to the microalgae, a culturing step (preculture) of growing the microalgae can be performed. Through the culturing step of growing the microalgae and the first step of applying the environmental stress to increase the accumulation of lipid, the lipid can be efficiently produced. The preculture can be performed through culturing the microalgae in a culture medium having an initial nitrogen concentration of 12 mg/L or more for three days or more.

The preculture performed at a high initial nitrogen concentration can significantly increase the cells of the algae. The environmental stress is applied to the algae that have been grown through the preculture, thereby increasing the lipid accumulated in the algae. As a result, the lipid can be produced more efficiently.

To increase the number of cells, the initial nitrogen concentration during the preculture is preferably 12 mg/L or more, more preferably 30 mg/L or more, still more preferably 40 mg/L or more, yet more preferably 50 mg/L or more, even more preferably 60 mg/L or more. To avoid excessive eutrophication of a culture solution, the initial nitrogen concentration is preferably 200 mg/L or less, more preferably 150 mg/L or less, still more preferably 100 mg/L or less, even more preferably 80 mg/L or less.

The initial nitrogen concentration for the preculture can be calculated by multiplying the concentration of a nitrogen-containing compound at the preparation of the culture solution with (molecular weight of nitrogen (g/mol)/molecular weight of the nitrogen-containing compound (g/mol)). For example, if sodium nitrate having a molecular weight of 85 g/mol is used as the nitrogen-containing compound, the initial nitrogen concentration can be calculated by multiplying the concentration of sodium nitrate with (14/85). The nitrogen concentration of the culture solution can be measured with a total nitrogen analyzer using an oxidative degradation-chemiluminescence method.

The culture solution used for the preculture may have a composition similar to that of a general culture medium for the algae. For example, the composition may be similar to that of f/2 medium, IMK medium, or ESM medium. Depending on the type of algae to be cultured, the culture medium may be selected and its composition may be adjusted as appropriate.

The culture solution for the preculture may contain salt. If the salt concentration of the culture solution for the preculture is increased to a certain degree, the accumulation of lipid can be increased in the subsequent first step of applying the environmental stress. To increase the accumulation of lipid, the salt concentration of the culture solution used for the preculture is preferably 18 g/L (1.8%) or more, more preferably 36 g/L (3.6%) or more, still more preferably 55 g/L (5.5%) or more, even more preferably 65 g/L (6.5%) or more. To sufficiently increase the cells during the preculture, the salt concentration of the culture solution for the preculture is preferably 85 g/L (8.5%) or less, more preferably 80 g/L (8.0%) or less. The salt concentration of the culture solution can be measured using a salinometer which measures the salt concentration from electric conductivity and liquid temperature.

The culture solution used for the preculture can be prepared by dissolving, for example, culture medium components necessary for the growth of the algae, such as a nitrogen component and any other components, in artificial seawater or artificial seawater added with salt to have a predetermined concentration. The culture medium components and a required amount of salt can be mixed in advance, and then the mixture can be dissolved in the artificial seawater. The artificial seawater may be replaced with natural seawater. Alternatively, the culture medium components, artificial seawater components, and a required amount of salt can be dissolved in water. The salt to be added can be sodium chloride.

The preculture can be performed until the algae are sufficiently grown. The culturing state is monitored while checking the cell count and the nitrogen concentration of the culture solution as indices, for example, so that the culturing continues until these values reach the predetermined values. Without monitoring the culturing state, only time can be managed.

Specific culture time can be selected depending on the conditions such as temperature, the amount of light irradiation, and the scale of the culture. In view of production efficiency, the culture time is preferably 3 days or more, more preferably 4 days or more, much more preferably 5 days or more, and preferably 21 days or less, more preferably 15 days or less, much more preferably 10 days or less.

In view of production efficiency, the concentration of the microalgae in the culture solution (algal concentration) at the onset of the preculture is preferably 0.01 g/L or more, more preferably 0.02 g/L or more, much more preferably 0.03 g/L or more, although it varies depending on the conditions and scale of the culture. The algal concentration at the end of the preculture, which can be selected depending on the culture environment or any other conditions, is preferably 0.1 g/L or more, more preferably 0.2 g/L or more, much more preferably 0.3 g/L or more.

In view of production efficiency, the nitrogen concentration at the end of the preculture is preferably 15 mg/L or less, more preferably 10 mg/L or less, much more preferably 5 mg/L or less.

### <First Step>

The first step is a step of culturing the microalgae (main culture) with an environmental stress being applied to increase the accumulation of lipid. For the main culture, the microalgae that have been grown through the preculture, for example, can be cultured in a culture medium having an initial nitrogen concentration of less than 12 mg/L for three days or more.

To increase the accumulation of lipid, the culture solution used for the main culture preferably has an initial nitrogen concentration of less than 12 mg/L, more preferably 6 mg/L or less, much more preferably 3 mg/L or less. The culture solution may be substantially free from a nitrogen component. In this context, the culture solution substantially free from a nitrogen component is a culture solution to which no nitrogen component has been intentionally added. Setting the nitrogen concentration low at the onset of the culture can increase the accumulation of lipid per cell.

The main culture can be performed with, for example, the culture solution that has been used for the preculture and diluted. To increase the production of lipid, the dilution ratio is preferably 1.6 or more, more preferably 2 or more, much more preferably 2.5 or more. To maintain the algal concentration to a certain level or more, the dilution ratio is preferably 20 or less, more preferably 15 or less, much more preferably 10 or less.

The algal concentration at the onset of the main culture is not particularly limited, and can be selected depending on the culture environment or any other conditions. To increase the production of lipid, the algal concentration is preferably 0.005 g/L or more, more preferably 0.01 g/L or more, much more preferably 0.015 g/L or more. In view of productivity of lipid, the algal concentration is preferably 3.0 g/L or less, more preferably 2.0 g/L or less, much more preferably 1.0 g/L or less.

A diluent for diluting the culture solution that has been used for the preculture is not particularly limited as long as the culture solution can be diluted to be suitable for the main culture. For example, the culture solution can be diluted to have a composition similar to that of a general culture medium used for culturing halotolerant algae, and a nitrogen concentration of less than 12 mg/L. Examples of the general culture medium used for culturing the halotolerant algae include f/2 medium, IMK medium, and ESM medium. The culture solution which has been used for the first culturing step and from which the nitrogen component has been removed may be used as the diluent. Alternatively, artificial seawater, artificial seawater diluted with water, or artificial seawater to which salt is added may also be used. The artificial seawater may be replaced with natural seawater. Water substantially free from salt may be used as the diluent.

If nitrogen in the culture solution has been consumed through the preculture and the nitrogen concentration of the culture solution is almost zero mg/L, the initial nitrogen concentration of the diluent can be set to less than 12 mg/L, preferably 10 mg/L or less, more preferably 5 mg/L or less in view of appropriate control of the nitrogen concentration at the onset of the main culture. The diluent may be substantially free from nitrogen.

The salt concentration of the diluted culture solution is not particularly limited as long as the microalgae can grow at that salt concentration. To increase the production of lipid, the salt concentration is preferably 90 g/L (9.0%) or less, more preferably 70 g/L (7.0%) or less, still more preferably 60 g/L (6.0%) or less, much more preferably 45 g/L (4.5%) or less, although it varies depending on the type of the microalgae. The salt concentration of the culture solution for the main culture can be reduced to the minimum level required for the growth of the algae. However, to increase the production of lipid, the salt concentration is preferably 15 g/L (1.5%) or more, more preferably 30 g/L (3.0%) or more. To increase the production of lipid, the salt concentration of the culture solution for the main culture is preferably 1.0 times or less, more preferably 0.9 times or less, much more preferably 0.8 times or less, higher than the salt concentration of the culture solution at the end of the preculture.

In view of efficient culture, a portion of the culture solution that has been used for the preculture is preferably withdrawn and diluted with a diluent for use in the main culture. If at least a portion of the culture solution that has been used for the preculture is withdrawn for use in the main culture, the preculture can be smoothly shifted to the main culture without separation of the algae and the culture solution. This can improve the production efficiency.

For example, after the preculture is performed in a first culture tank, a portion of the culture solution can be transferred from the first culture tank to a second culture tank, and diluted with a diluent at a predetermined dilution ratio for use in the main culture. Additional culture solution can be poured into the first culture tank for passage culture. In this manner, the maintenance of the algae strain and the production of lipid can be efficiently done. Alternatively, the culture solution may be transferred from the first culture tank to some culture tanks, in each of which the main culture can be performed. To keep the algal concentration to a certain level or higher, preferably 5 vol % or more, more preferably 10 vol % or more, still more preferably 15 vol % or more, much more preferably 20 vol % or more, of the culture solution can be transferred to the second culture tank. To increase the accumulation of lipid, preferably 60 vol % or less, more preferably 50 vol % or less, still more preferably 40 vol % or less, much more preferably 30 vol % or less, of the culture solution can be transferred. Alternatively, the diluent may be added to the first culture tank to perform the main culture, and passage culture may be performed in the second culture tank into which a portion of the culture solution has been transferred.

The main culture is performed until a sufficient amount of lipid is accumulated in the cells of the algae. To increase the production of lipid, the accumulation of lipid in the algae at the end of the main culture is preferably 35% or more, more preferably 40% or more, still more preferably 45% or more, much more preferably 50% or more. In view of productivity of lipid, the accumulation of lipid is preferably 80% or less, more preferably 70% or less, much more preferably 65% or less. The accumulation of lipid in the algae can be obtained by a method described in the examples.

Through monitoring how much the lipid is accumulated in the algae based on an index except for the accumulation of lipid, the algae can be cultured until the value being monitored corresponds to a predetermined condition. Without monitoring the culturing state, only time can be managed. In general, time for the main culture can be selected depending on the conditions such as temperature, the amount of light irradiation, and the scale of the culture. In view of production efficiency, the culture time is preferably 3 days or more, more preferably 5 days or more, still more preferably 10 days or more, much more preferably 15 days or more, and preferably 40 days or less, more preferably 30 days or less, much more preferably 25 days or less.

The preculture and the main culture can be performed at a temperature at which the algae can grow. For example, the temperature can be selected within the range of 5° C. to 40° C. In view of efficient culture, the temperature is preferably 10° C. to 35° C., more preferably 15° C. to 30° C., much more preferably 20° C. to 30° C. The temperature control may be actively performed or not in view of the size of the culture tank, the environment in which the culture tank is placed, the efficiency of the culture required, and costs.

The preculture and the main culture can be performed by making use of solar light. Alternatively, artificial light may be used, or the solar light and the artificial light may be used in combination. During the culture, a light period and a dark period may be produced at a predetermined cycle. In the first and second culturing steps, the content of the culture tank may be actively stirred, or natural convection may be provided in the tank. Alternatively, the culture solution may be bubbled to increase the amount of air or carbon dioxide dissolved in the culture solution.

As the first step of applying an environmental stress, a culturing step performed in the nitrogen-deficient state has been described. However, in place of or in addition to the nitrogen-deficient state, other environmental stresses, e.g., the salt concentration, ultraviolet rays, culture density, and culture temperature, may be added as long as the lipid can be accumulated in the cells of the microalgae.

### <Second Step>

In the second step, the microalgae that have received the environmental stress and increased the accumulation of lipid in the algae are thermally treated at a pH of 3.5 or more and 9.5 or less and a temperature of 40° C. or more and 65° C. or less. Through the thermal treatment performed at the pH and temperature in the predetermined ranges, the recovery of lipid from the microalgae that have had the environmental stress can be as easy as, or easier than, the recovery from the microalgae with no environmental stress.

To improve the recovery rate of lipid through the efficient treatment of the microalgae, the thermal treatment is performed at a pH of 2.0 or more, preferably 3.0 or more, more preferably 3.5 or more, much more preferably 3.8 or more, even more preferably 4.0 or more, far more preferably 4.5 or more, yet more preferably 5.0 or more. Further, to improve the recovery rate of lipid through the efficient treatment of the microalgae, the pH is 10.0 or less, preferably 9.5 or less, more preferably 9.0 or less, much more preferably 8.5 or less, even more preferably 8.0 or less, still more preferably 7.5 or less, far more preferably 7.0 or less, a lot more preferably 6.5 or less, yet more preferably 6.0 or less.

The pH may be measured in a treatment solution at 25° C. by a method in conformity with JIS Z8802.

To improve the recovery rate of lipid through the efficient treatment of the microalgae, the temperature for the thermal treatment is 35° C. or more, preferably 40° C. or more, more preferably 42° C. or more, still more preferably 43° C. or more, yet more preferably 45° C. or more. Further, to improve the recovery rate of lipid through the efficient treatment of the microalgae, the temperature is 80° C. or less, preferably 65° C. or less, more preferably 62° C. or less, even more preferably 60° C. or less, still more preferably 57° C. or less, yet more preferably 55° C. or less, a lot more preferably 52° C. or less, much more preferably 50° C. or less.

The thermal treatment time is a period during which the temperature of the treatment solution is maintained within the above-described temperature range. To improve the recovery rate of lipid through the efficient treatment of the microalgae, the treatment time is preferably 0.5 hours or more, more preferably 1 hour or more, much more preferably 3 hours or more, even more preferably 5 hours or more, still more preferably 8 hours or more, far more preferably 10 hours or more, yet more preferably 20 hours or more, a lot more preferably 24 hours or more, a great deal more preferably 48 hours or more. In view of production efficiency, the upper limit of the treatment time is preferably 96 hours or less, more preferably 72 hours or less.

The thermal treatment may be continuously performed for a predetermined time, or performed in several steps such that the sum of the treatment times of these steps is the predetermined time.

The pH, the temperature, and the time respectively within the above-described ranges may be combined as appropriate. In particular, to improve the recovery rate of lipid through the efficient treatment of the microalgae, the pH is preferably 5.0 or more and 7.5 or less, the temperature is preferably 45° C. or more and 60° C. or less, and the treatment time is preferably 10 hours or more. Setting the pH and the temperature within the above-described ranges activates an enzyme in the algal cell, thereby promoting the decomposition of the cell wall.

The thermal treatment may be performed in an open or closed treatment tank. The temperature in the treatment tank may be controlled by a generally known method. For example, a heat source, and a controller which turns the heat source on or off to control the temperature in the tank to a predetermined level may be provided. The temperature may be controlled at a general industrial precision level, for example, within a tolerance of ±5° C. or less, preferably ±3° C. or less, more preferably ±1° C. or less. If the temperature cannot be controlled easily, it is sufficient for the sum of the periods during which the temperature of the treatment solution is in a predetermined temperature range to be the predetermined time. The heat source may be arranged inside or outside the treatment tank. The treatment tank may be a batch type tank or a flow type tank. If the flow type tank is used, the tank may have the shape of a passage through which the treatment solution flows. The thermal treatment may be performed at normal pressure, or in a pressurized or depressurized environment.

The treatment temperature may be measured with, for example, a thermometer or temperature sensor inserted in the treatment solution. Alternatively, a noncontact temperature sensor may be used to measure the temperature of the treatment solution. An ambient or external temperature of the treatment tank may be measured instead of directly measuring the temperature of the treatment solution. In such a case, a correlation coefficient between the ambient or external temperature and the temperature of the treatment solution is obtained in advance, and the ambient or external temperature may be converted into the temperature of the treatment solution. Alternatively, the output of the thermometer or temperature sensor may be sent to a recorder to record the temperature continuously or periodically such that the thermal treatment time is controlled with high precision.

The thermal treatment can be directly performed on the culture solution that has been used for the main culture. The thermal treatment can be performed after the culture solution is diluted or concentrated for adjusting the algal concentration. In addition, a dispersion medium may be replaced, or an additive may be added.

The algal concentration of the treatment solution is not particularly limited. In view of treatment efficiency and recovery of lipid, the algal concentration is preferably 0.1 g/L or more, more preferably 0.5 g/L or more, even more preferably 0.8 g/L or more, yet more preferably 1 g/L or more, still more preferably 5 g/L or more. In view of flowability of the treatment solution and the recovery of lipid, the algal concentration is preferably 300 g/L or less, more preferably 250 g/L or less, still more preferably 200 g/L or less, even more preferably 100 g/L or less, yet more preferably 50 g/L or less, far more preferably 20 g/L or less, a lot more preferably 10 g/L or less. The algal concentration of the treatment solution is measured by a method described in the examples.

Although it is not particularly limited, in view of treatment efficiency and recovery of lipid, the second step is performed on the algae in which the accumulation of lipid is preferably 35% or more, more preferably 40% or more, still more preferably 45% or more, much more preferably 50% or more, and preferably 80% or less, more preferably 70% or less, much more preferably 65% or less.

If the culture solution is diluted or the dispersion medium is replaced, a diluent or a replacement solution is not particularly limited as long as the pH of the culture solution is within the predetermined range, but water is preferably used in view of cost efficiency. Seawater may also be used. The diluent or the replacement solution may contain any one or more of additives including salt such as sodium chloride, a compound containing nitrogen or phosphorus, trace metal, an inorganic flocculant, an organic flocculant, a chelating agent, and a buffer. The finally prepared treatment solution may contain various components that are contained in a general culture solution.

The culture solution can be concentrated through, for example, filtration, pressing, centrifugation, gravity sedimentation, flocculated sedimentation, floatation, or evaporation of a dispersion medium. These techniques may be combined for multistage concentration.

If the pH of the treatment solution is different from the predetermined value, acid or alkali may be added to the treatment solution to adjust the pH. Any kinds of acid may be used without particular limitation, and organic acids, mineral acids, or a mixture of these acids may be used. For example, acetic acid, citric acid, phosphoric acid, hydrochloric acid, nitric acid, or sulfuric acid may be used. Any kinds of alkali may be used without particular limitation, and sodium carbonate, ammonia, or sodium hydroxide may be used. A buffer solution may be used as a dispersion medium. The buffer solution may be selected depending on the pH required. For example, a buffer solution containing acetic acid, citric acid, phosphoric acid, sodium carbonate, or any other suitable component may be used.

During the thermal treatment, any additive may be added to the treatment solution. However, it is not necessary to actively add enzymes having the action of decomposing cell walls, such as hemicellulase, cellulase, pectinase, and laminarinaze. Further, it is not necessary to actively add agents having the action of decomposing cell walls, such as salt, alkali, a surfactant, and a detergent. Note that these enzymes or agents may be contained in the treatment solution.

The treatment solution can be subjected to the thermal treatment just after the preparation, may be preserved at low or normal temperature, or cryopreserved. It is preferred that the microalgae have not been exposed to a temperature of 80° C. or more before the thermal treatment.

### <Process of Recovery of Lipid>

From the algae which have received the environmental stress to increase the accumulation of lipid, and then have been thermally treated according to this embodiment, the lipid is recovered. As a result, the lipid can be efficiently recovered from the algae.

### —Lipid—

In the context of the present disclosure, the lipid may include simple lipid, complex lipid, and derived lipid. The simple lipid may include fats and oils, and esters of fatty acid and various types of alcohols, such as fatty acid ester. The complex lipid may include phospholipid containing fatty acid, alcohol and phosphoric acid, and glycolipid containing fatty acid, alcohol and sugar. The derived lipid may include water-insoluble fatty acid, higher alcohol, sterol, terpene, and fat-soluble vitamins, which are products of hydrolysis of the simple or complex lipid. In view of recovery of lipid, the simple or complex lipid is preferable, the simple lipid is more preferable, and fats and oils are much more preferable.

Fats and Oils

Fats and oils designate esters of fatty acid and glycerin, in particular, neutral lipid such as monoglyceride, diglyceride, and triglyceride. The fatty acid constituting the fats and oils is not limited, and may include various kinds of fatty acids.

Fatty Acid

Fatty acid may be any of short-chain fatty acids having a carbon number of 2 to 4, medium-chain fatty acids having a carbon number of 5 to 12, and long-chain fatty acids having a carbon number of 12 or more. The fatty acid may be saturated or unsaturated. Examples of the saturated fatty acid may include decanoic acid, dodecanoic acid, tetradecanoic acid, hexadecanoic acid, octadecanoic acid, and icosanoic acid. Examples of monounsaturated fatty acid may include 9-hexadecenoic acid and 9-octadecenoic acid. Examples of polyunsaturated fatty acid may include 9,12-octadecadienoic acid, 6,9,12-octadecatrienoic acid, 5,8,11,14-icosatetraenoic acid, 9,12,15-octadecatrienoic acid, 5,8,11,14,17-icosapentaenoic acid, and 4,7,10,13,16,19-docosahexaenoic acid.

Fatty Acid Esters

Fatty acid esters are esters of fatty acid and alcohol, except for fats and oils, and may include wax which is an ester of long-chain fatty acid and higher monohydric or dihydric alcohol, and a medium-chain fatty acid ester which is an ester of medium-chain fatty acid and lower or higher alcohol.

### —Recovery of Lipid—

A process of the lipid recovery is not particularly limited as long as the lipid is separated from the microalgae that have gone through the second step. For example, the lipid recovery may be achieved by solvent extraction, centrifugation, flocculated sedimentation, standing, column chromatography, or any other suitable technique. One or a combination of two or more of these techniques may be adopted. Above all, in view of recovery of lipid, one or a combination of two or more of solvent extraction, centrifugation, flocculated sedimentation, and standing is preferable. In particular, a combination of solvent extraction and centrifugation, or a combination of solvent extraction and standing is preferable.

For the solvent extraction, a solution for extraction may be added to, and mixed with, the solution containing the microalgae that have gone through the second treatment. The mixture thus obtained may be stirred. Lipid eluted from the microalgae dissolves in the solvent. Thus, a solvent phase and an aqueous phase are separated to recover the solvent phase, thereby yielding the lipid.

Examples of the solvent used for the solvent extraction include: esters such as methyl acetate and ethyl acetate; chain and cyclic ethers such as tetrahydrofuran and diethyl ether; polyethers such as polyethylene glycol; halogenated hydrocarbons such as dichloromethane, chloroform, and carbon tetrachloride; hydrocarbons such as hexane, cyclohexane, and petroleum ether; aromatic hydrocarbons such as benzene and toluene; pyridines; alcohols such as butanol, pentanol, hexanol, and isopropyl alcohol (2-propanol); polyalcohols such as butylene glycol; ketones such as methyl ethyl ketone; and supercritical carbon dioxide. One or a combination of two or more of these substances may be used.

Among these solvents, nonpolar solvents are preferably used in view of recovery of lipid. Examples of the nonpolar solvents may include halogenated hydrocarbons, hydrocarbons, and aromatic hydrocarbons. In particular, the hydrocarbons are preferable, among which hexane is more preferable. Further, a solvent compatible to water, such as methanol, ethanol, propanol, ethylene glycol, propylene glycol, and acetone, may be supplementarily added to the nonpolar solvent. Moreover, supercritical extraction using supercritical carbon dioxide may also be adopted. In addition, immersion, decoction, leaching, reflux extraction, subcritical extraction, or any other technique may also be adopted. For example, a method described in “Biochemical Experimentation Method 24—Method of Experimenting Lipid Metabolism in Plants” (Akihiro YAMADA, Japan Scientific Societies Press, pp. 3-4) can also be referred to.

The solvent extraction may be performed at any temperature without particular limitation. However, in view of recovery of lipid, the temperature is preferably 10° C. or more, more preferably 20° C. or more. In view of recovery of lipid and cost of heating the solvent, the temperature is preferably 60° C. or less, more preferably 50° C. or less, much more preferably 40° C. or less.

The solvent extraction may be performed once, or twice or more. If the solvent extraction is performed twice or more, it may be performed with the same solvent or different solvents.

The centrifugation may be performed with a generally known apparatus such as a disk centrifuge, a cylindrical centrifuge, a decanter centrifuge, or any other type of centrifuge. In this case, a centrifugal force may preferably be 500 G or more, more preferably 1000 G or more, in view of recovery of lipid. Further, in view of cost efficiency, the centrifugal force may preferably be 10000 G or less, more preferably 5000 G or less, much more preferably 2000 G or less.

The centrifugation may preferably be performed for one minute or more, more preferably five minutes or more, much more preferably 10 minutes or more in view of recovery of lipid. Further, in view of cost efficiency, the centrifugation time may preferably be 80 minutes or less, more preferably 40 minutes or less, much more preferably 20 minutes or less.

The centrifugation may be performed at any temperature without particular limitation. However, in view of recovery of lipid and cost efficiency, the temperature is preferably 10° C. or more, more preferably 15° C. or more, and preferably be 50° C. or less, more preferably 40° C. or less.

If the solvent extraction and the centrifugation are combined, a solvent phase and an aqueous phase can be separated quickly by the centrifugation.

During the standing process, the reaction solution is allowed to stand until lipid and an aqueous phase are separated. If the standing process and the solvent extraction are combined, the reaction solution may be allowed to stand until a solvent phase and an aqueous phase are separated.

The disclosed method for recovering the lipid from the algae makes it possible to recover the lipid, which has been accumulated at high concentration in the microalgae through the application of an environmental stress, with high yield by such a simple operation.

The lipid extracted from the microalgae may be used directly, or indirectly after purification or decomposition, as biofuels such as a biodiesel fuel. Further, the lipid may also be used as materials for functional food, pharmaceuticals, chemical products, and cosmetics.

The lipid recovered and produced from the microalgae generally contains fats and oils. Using the fats and oils as raw materials, aliphatic alcohol and/or glycerin can be produced by the following processes [A] to [E]. The fats and oils used as the raw materials may be those separated in advance from the lipid, or those being contained in the lipid.

[A: Production of Aliphatic Alcohol and/or Glycerin from Fats and Oils]

Fats and oils obtained from the microalgae are reduced through hydrogenation in the presence of a catalyst to produce aliphatic alcohol and/or glycerin derived from an inedible material that does not compete with food. The catalyst used for the hydrogenation is not particularly limited, and may be a hydrogenation catalyst used for generally known alcohol production. Examples of the hydrogenation catalyst include: Co-based catalysts such as a Co/Mo catalyst and a Co/Zr catalyst; Cu-based catalysts such as a Cu/Cr catalyst and a Cu/Zr catalyst; Re-based catalysts; Ru-based catalysts; Rh-based catalysts; and noble metal-based catalysts such as platinum catalysts. Among these catalysts, the Ru-based catalysts and the Co-based catalysts are preferable, the Co-based catalysts are more preferable, and in particular, a Co/Zr catalyst is even more preferable. In a preferred embodiment, the hydrogenation is performed with the coexistence of water so that the yield of glycerin increases.

[B: Production of Aliphatic Alkyl Ester and/or Glycerin from Fats and Oils]

Transesterification is performed between fats and oils obtained from the microalgae and lower alcohol having a carbon number of 1 to 5 in the presence of a catalyst to produce aliphatic alkyl ester and/or glycerin. The catalyst used for the transesterification may be homogeneous or heterogeneous. Examples of the homogeneous catalyst include sodium hydroxide, potassium hydroxide, and sodium alcoholate. Examples of the heterogeneous catalyst include ion exchange resins, calcium oxide, magnesium oxide, polymer compounds having an amino group or an ammonium group on a side chain, inorganic phosphate, and weak acid catalysts containing organic phosphate and one or more metal atom(s) selected from aluminum, gallium, and iron. Among these catalysts, weak acid heterogeneous catalysts are preferably used because they can be used at high reaction temperature and do not generate by-products such as soap.

[C: Production of Aliphatic Alcohol from Aliphatic Alkyl Ester]

Aliphatic alkyl ester obtained in the process [B] is reduced through hydrogenation in the presence of a catalyst to produce aliphatic alcohol derived from an inedible material that does not compete with food. The catalyst used for the hydrogenation is not particularly limited, and may be a hydrogenation catalyst used for generally known alcohol production. Examples of the catalyst include: Cu-based catalysts such as Cu/Cr, Cu/Zn, Cu/Fe, Cu/Al, and Cu/Si catalysts; Co-based catalysts such as Co/Mo and Co/Zr catalysts; Re-based catalysts; Ru-based catalysts; Rh-based catalysts; and noble metal-based catalysts such as platinum catalysts. Among these catalysts, the Cu-based catalysts are preferable, in particular, the Cu/Zn catalyst is more preferable.

[D: Production of Fatty Acid and/or Glycerin from Fats and Oils]

Fats and oils obtained from the microalgae are hydrolyzed to produce fatty acid and/or glycerin. The hydrolysis can be performed by high pressure decomposition, medium pressure decomposition, enzymolysis, or any other suitable techniques.

[E: Production of Aliphatic Alcohol from Fatty Acid]

Fatty acid obtained in the process [D] is reduced through hydrogenation in the presence of a catalyst to produce aliphatic alcohol derived from an inedible material that does not compete with food. The catalyst used for the hydrogenation is not particularly limited, and may be a hydrogenation catalyst used for generally known alcohol production. Examples of the catalyst include: Co-based catalysts such as a Co/Mo catalyst and a Co/Zr catalyst; Cu-based catalysts such as Cu/Cr, Cu/Zn, Cu/Fe, Cu/Al, and Cu/Si catalysts; Re-based catalysts; Ru-based catalysts; Rh-based catalysts; and noble metal-based catalysts such as platinum catalysts.

As can be seen, the process of recovering the lipid from the microalgae that have been obtained through the treatment of this embodiment, and the method of producing aliphatic alcohol and/or glycerin from fats and oils contained in the lipid have been described. However, products other than the lipid, such as sugar and protein, may also be recovered for use. These products can be recovered by a generally known method.

Regarding the above-described embodiment, the present disclosure further discloses an algae treatment method described below.

<1>

An algae treatment method including: a first step of culturing microalgae belonging to Heterokontophyta in a culture medium having an initial nitrogen concentration of less than 12 mg/L, preferably 10 mg/L or less, more preferably 5 mg/L or less for 3 days or more, preferably 5 days or more, and preferably 21 days or less, more preferably 15 days or less, much more preferably 10 days or less; and a second step of performing a thermal treatment of the microalgae that have gone through the first step at a pH of 2.0 or more, preferably 3.0 or more, more preferably 3.5 or more, much more preferably 3.8 or more, even more preferably 4.0 or more, far more preferably 4.5 or more, yet more preferably 5.0 or more, and 10.0 or less, preferably 9.5 or less, more preferably 9.0 or less, much more preferably 8.5 or less, even more preferably 8.0 or less, still more preferably 7.5 or less, a lot more preferably 6.5 or less, yet more preferably 6.0 or less, and a temperature of 35° C. or more, preferably 40° C. or more, more preferably 42° C. or more, still more preferably 43° C. or more, yet more preferably 45° C. or more, and 80° C. or less, preferably 65° C. or less, more preferably 62° C. or less, even more preferably 60° C. or less, still more preferably 57° C. or less, yet more preferably 55° C. or less, a lot more preferably 52° C. or less, much more preferably 50° C. or less.

<2>

The algae treatment method of <1>, wherein the microalgae are of the genus Nannochloropsis.

<3>

The algae treatment method of <1> or <2>, wherein the thermal treatment is performed for 0.5 hours or more, preferably 1 hour or more, more preferably 3 hours or more, even more preferably 5 hours or more, still more preferably 8 hours or more, far more preferably 10 hours or more, yet more preferably 20 hours or more, a lot more preferably 24 hours or more, a great deal more preferably 48 hours or more, and preferably 96 hours or less, more preferably 72 hours or less.

<4>

The algae treatment method of any one of <1> to <3>, further including: prior to the first step, a culturing step of culturing the microalgae in a culture medium having an initial nitrogen concentration of 12 mg/L or more, more preferably 30 mg/L or more, still more preferably 40 mg/L or more, yet more preferably 50 mg/L or more, even more preferably 60 mg/L or more, and 200 mg/L or less, more preferably 150 mg/L or less, still more preferably 100 mg/L or less, even more preferably 80 mg/L or less, for 3 days or more, more preferably 4 days or more, much more preferably 5 days or more, and preferably 21 days or less, more preferably 15 days or less, much more preferably 10 days or less.

<5>

The algae treatment method of <4>, wherein the first step is performed using the culture solution that has been used for the culturing step and diluted 1.6-fold or more, preferably 2-fold or more, more preferably 2.5-fold or more, and 20-fold or less, preferably 15-fold or less, more preferably 10-fold or less.

<6>

The algae treatment method of <4> or <5>, wherein the salt concentration of the culture solution for the first step is preferably 1.0 times or less, more preferably 0.9 times or less, much more preferably 0.8 times or less, higher than the salt concentration of the culture solution at the end of the culture.

<7>

The algae treatment method of any one of <4> to <6>, wherein the first step is performed using 5 vol % or more, preferably 10 vol % or more, more preferably 15 vol % or more, much more preferably 20 vol % or more, and 60 vol % or less, preferably 50 vol % or less, more preferably 40 vol % or less, much more preferably 30 vol % or less, of the culture solution that has gone through the culturing step.

<8>

The algae treatment method of any one of <1> to <7>, wherein the second step is performed at an algal concentration of 0.1 g/L or more, preferably 0.5 g/L or more, more preferably 0.8 g/L or more, yet more preferably 1 g/L or more, still more preferably 5 g/L or more, and 300 g/L or less, preferably 250 g/L or less, more preferably 200 g/L or less, even more preferably 100 g/L or less, yet more preferably 50 g/L or less, far more preferably 20 g/L or less, a lot more preferably 10 g/L or less.

<9>

The algae treatment method of any one of <1> to <8>, wherein the second step is performed on the algae in which the accumulation of lipid is preferably 35% or more, more preferably 40% or more, still more preferably 45% or more, much more preferably 50% or more, and preferably 80% or less, more preferably 70% or less, much more preferably 65% or less.

<10>

The algae treatment method of any one of <1> to <9>, wherein the microalgae are grown in the culturing step, and lipid is accumulated in the microalgae in the first step.

<11>

A method of producing lipid, the method including: recovering the lipid from the algae treated through the method of any one of <1> to <10>.

<12>

The method of <11>, wherein the lipid contains fats and oils.

<13>

A method of producing aliphatic alcohol and/or glycerin, the method including: performing hydrogenation of the fats and oils obtained through the method of <12> in the presence of a catalyst.

<14>

A method of producing aliphatic alkyl ester and/or glycerin, the method including: performing transesterification of the fats and oils produced through the method of <12> with lower alcohol having a carbon number of 1 to 5 in the presence of a catalyst.

<15>

A method of producing aliphatic alcohol, the method including: performing hydrogenation of the aliphatic alkyl ester produced through the method of <14> in the presence of a catalyst.

<16>

A method of producing fatty acid and/or glycerin, the method including: hydrolyzing the fats and oils produced through the method of <12>.

<17>

A method of producing aliphatic alcohol, the method including: performing hydrogenation of the fatty acid produced through the method of <16>.

### EXAMPLES

The present disclosure will be described in further detail by way of examples. Examples described below are merely exemplary ones, and do not limit the present invention.

### <Algae>

A seawater sample containing algae was collected from a coastal region of Ishigaki Island, Okinawa. The seawater sample thus collected was concentrated with a filter, and a single algal strain was isolated with a micropipette. The algae thus isolated were cultured in a culture solution (Daigo's IMK medium of Wako Pure Chemical Industries, Ltd.), and grown using a culture solution (f/2 medium). As a result of analysis of part of the algae by University of Texas (UTEX Culture Collection), the algae were identified as Nannochloropsis salina.

### <Culture Solution>

The culture solution was prepared in accordance with f/2 medium. Table 1 shows the composition of the culture solution per 100 mL. Table 2 shows the compositions of trace metals (f/2 metals) shown in Table 1. As the solvent, artificial seawater (Daigo's artificial seawater SP manufactured by Wako Pure Chemical Industries, Ltd.), or salt-added artificial seawater of a predetermined salt concentration prepared by adding sodium chloride (manufactured by Wako Pure Chemical Industries, Ltd.) to the artificial seawater was used. The artificial seawater used had a salt concentration of 36 g/L (3.6%). The salt concentration of the culture solution was measured with a salinometer (PAL-06S manufactured by Atago Co., Ltd.).

TABLE 1

| Components | Amount |
|---|---|
| NaNO₃ | Set amount |
| NaH₂PO₄•2H₂O | Set amount |
| Vitamin B12 | 0.05 μg |
| Biotin | 0.05 μg |
| Thiamine HCl | 10 μg |
| Trace metal (f/2 metals) | 0.1 mL |
| Solvent | 99.9 mL |

TABLE 2

| Components | Amount |
|---|---|
| NaEDTA•2H₂O | 440 mg |
| FeCl₃•6H₂O | 316 mg |
| CoSO₄•7H₂O | 1.2 mg |
| ZnSO₄•7H₂O | 2.1 mg |
| MnCl₂•4H₂O | 18 mg |
| CuSO₄•5H₂O | 0.7 mg |
| Na₂MoO₄•2H₂O | 0.7 mg |
| Distilled water | 100 mL |

### <Diluent>

As the diluent, artificial seawater (Daigo's artificial seawater SP manufactured by Wako Pure Chemical Industries, Ltd.) having a salt concentration of 36 g/L (3.6%), or salt-added artificial seawater of a predetermined salt concentration prepared by adding sodium chloride (manufactured by Wako Pure Chemical Industries, Ltd.) to the artificial seawater was used. The salt concentration of the diluent was measured with a salinometer (PAL-06S manufactured by Atago Co., Ltd.).

### <Culture Conditions>

A predetermined amount of a culture solution was placed in a polycarbonate bottle having a diameter of 25 cm and a height of 25 cm to provide a sterilized closed system. Air containing 1.5% of CO₂ was blown through the culture solution using a ceramic airstone (manufactured by IBUKI airstone, 30Ø×78, #100). An LED square light (LED-12-40DA manufactured by Electric Lightening LED System Co., Ltd.) was used as a light source to laterally irradiate the bottle with light. The irradiation with light was performed in a 24-hour cycle of keeping the light source on for 12 hours and keeping the light source off for 12 hours. The light intensity measured on the side of the bottle with a light intensity quantum meter (MQ-200 of Apogee Instruments) was 2000 μmol/m²/s (measured as a photon flux density (μmol/m²/s) at a wavelength of 409 to 659 nm). The temperature of a culture room was set to be 25° C.

### <Measurement of Nitrogen Amount>

A dispersion containing the microalgae was vacuum-filtrated with a filter (Supor-450 of Pall Corporation, pore size: 0.45 μm). With a total nitrogen analyzer (TN-2100V of Mitsubishi Chemical Analytech Co., Ltd.), 0.5 mL of the filtrate was analyzed.

### <Measurement of Algal Concentration>

A dispersion containing the microalgae was vacuum-filtrated with a filter (Supor-450 of Pall Corporation, pore size: 0.45 μm), and washed with an equal amount of distilled water. The filter that had trapped the algal bodies was moved into an aluminum cup, and dried at 105° C. for 2 hours. The weight of the dried filter was measured, the tare of the filter was subtracted from the measured weight to obtain dry weight (g). A value obtained by dividing the dry weight by the volume (L) of the dispersion used was regarded as the algal concentration (g/L). If the dispersion was not easily vacuum-filtrated with the filter, the dispersion was diluted to a suitable concentration with the artificial seawater (Daigo's artificial seawater SP manufactured by Wako Pure Chemical Industries, Ltd.), the weight of the dried filter was measured, the tare of the filter was subtracted from the measured weight, and then the obtained value was multiplied by the dilution ratio to obtain the algal concentration (g/L).

### <Thermal Treatment>

To the culture solution used for the main culture, 1 M hydrochloric acid or 1 M sodium hydroxide was dropped to adjust the pH to a predetermined value. The culture solution with the adjusted pH was placed in a polyethylene container, and left stand in a chamber of an automatic oven (ADP300 of Yamato Scientific Co., Ltd.) preset at a predetermined temperature. The pressure in the chamber was set to an atmospheric pressure. The temperature of the treatment solution was measured with a thermocouple (TM20 of Yokogawa Meters & Instruments) to confirm that the treatment solution had a predetermined temperature. After the lapse of a predetermined time during which the treatment solution maintained at the predetermined temperature, the sample was removed from the oven, and cooled at room temperature.

### <Measurement of Amount of Lipid Recovery>

The amount of lipid recovered from the algae was analyzed by a method for lipid extraction from biological materials reported by E. G. Bligh and W. J. Dyer in 1959 (Bligh & Dyer Method) (E. G. Bligh, W. J. Dyer, Canadian journal of biochemistry and physiology, 37 (1959), pp. 911-917).

To 0.5 mL of the culture solution with the algae dissolved therein, 100 μL of 1 mg/mL 7-pentadecanone (a methanol solution) was added as an internal standard. Then, 10 μL of 2 N HCl was added, and 500 μL of chloroform and 0.9 mL of methanol were added. After being stirred, the obtained solution was left stand at 25° C. for 30 minutes, and 500 μL of chloroform and 500 μL of 1.5% KCl were added. After being stirred, the obtained solution was centrifuged (centrifugal force: 1500 G, number of revolutions: 3000 r/min, temperature: 25° C., time: 15 minutes) with a centrifuge “himac CF7D2” (Hitachi Koki Co., Ltd.). Then, a lower chloroform phase was collected, and dried and solidified with nitrogen. Subsequently, 0.7 mL of a 0.5 N KOH-methanol solution (2.8 g of potassium hydroxide, 100 mL of methanol) was added, incubated at 80° C. for 30 minutes for saponification. Further, 1 mL of a 14% boron trifluoride solution was added, and incubated at 80° C. for 10 minutes for methyl esterification. Then, 1 ml of a solvent and 1 ml of saturated brine were added, stirred, and left stand at 25° C. for 30 minutes to obtain a solvent phase. Hexane was used as the solvent. The solvent phase thus obtained was recovered, and fatty acid ester was identified and quantified by gas chromatography (GC) under the following conditions. The identification of the fatty acid ester was performed by determining whether the retention time of the fatty acid ester was identical to that of a reference material to be described later. The amount of the fatty acid ester detected through the GC analysis was calculated relative to the internal standard, and the total amount was obtained as the lipid content (mg) in 0.5 mL of the culture solution. The obtained value was divided by 0.5 mL, which is the volume of the culture solution used for the analysis, to obtain the amount of lipid recovered (g/L) from one liter of the culture solution. From the algal concentration (g/L) obtained by the method described above, the amount of lipid recovery per g of algae was obtained.

The amount of lipid recovery per g of algae was also obtained from a culture solution treated with alkali. For the alkali treatment, 238 μl of an aqueous solution of sodium hydroxide having a concentration of 48% was added to 0.5 mL of the culture solution, and the mixture was maintained at 80° C. for two hours. Thereafter, 1 mL of 5N hydrochloric acid was added, and extraction and quantification of the lipid were performed.

The accumulation of lipid in the algae (%) can be obtained from the algal concentration (g/L) obtained by the method described above based on the following formula (1).

Accumulation of lipid in algae (%)=Amount of lipid recovered from one liter of culture solution treated with alkali (g/L)/algal concentration (g/L)×100 (1)

### —GC Analysis—

Apparatus: Agilent technology 7890A

Column: DB1-MS (product of J&W Scientific, 20 m×100 μm×0.1 μm)

Furnace Temperature: 150° C. (0.5 min hold)—[40° C./min]—220° C. (0 min hold) —[20° C./min]—320° C. (2 min hold)—post run 2 min

Carrier Gas: Hydrogen

Makeup Gas: Helium

Amount of Sample Injection: 5 μL

Injection Mode: Split (Sprit Rate=75:1)

Injection Port Temperature: 300° C.

Detector: FID

Flow Rate in Column: 0.28 mL/min, Constant

Pressure (Gauge Pressure): 62.403 psi

Reference Material: the following fatty acid esters available from SIGMA: methyl laurate (C12), methyl myristate (C14), methyl palmitate (C16), methyl stearate (C18), methyl palmitoleate (C16:1), methyl oleate (C18:1), methyl linoleate (C18:2), methyl linolenate (C18:3), methyl eicosapentaenoate (C20:5), methyl docosahexaenoate (C22:6)

### Example 1

### —Preculture—

As the microalgae belonging to Heterokontophyta, Nannochloropsis salina was dispersed in 10 L of a culture solution having a salt concentration of 36 g/L (3.6%). At the onset of the culture, the culture solution had an initial sodium nitrate concentration of 369 mg/L (60.8 mg/L as the initial nitrogen concentration), and the concentration of sodium dihydrogen phosphate dihydrate was 30 mg/L. The algal concentration after 10-day culture was 0.90 g/L.

### —Main Culture—

Of 10 L of the culture solution that had gone through the preculture, 2 L (20% of the culture solution) was placed in a polycarbonate bottle. To the culture solution, 8 L of salt-added artificial seawater having a salt concentration of 36 g/L (3.6%) was added as a diluent (dilution ratio: 5), and the main culture was started and continued for 24 days. The initial nitrogen concentration at the onset of the main culture was 0.9 mg/L, and the algal concentration at the end of the main culture was 1.03 g/L.

### —Thermal Treatment—

The pH of the culture solution after the main culture was adjusted to 5.0 with 1M HCl, and then the culture solution was maintained at 50° C. for 72 hours. The amount of lipid recovery per g of algae was 0.53 g. The amount of lipid recovery per g of algae when an alkali treatment was performed was 0.53 g.

### Comparative Example 1

The process was performed in the same manner as in Example 1 except that the thermal treatment was omitted. The algal concentration at the end of the main culture was 1.03 g/L, and the amount of lipid recovery per g of algae was 0.26 g. The amount of lipid recovery per g of algae when an alkali treatment was performed was 0.52 g.

### Comparative Example 2

Using the culture solution that had been used for the preculture as the diluent for the main culture, the main culture was performed with no environmental stress applied. The main culture was performed for 25 days. Other conditions were the same as those of Example 1. The initial nitrogen concentration at the onset of the main culture was 54.9 mg/L, and the algal concentration at the end of the main culture was 1.89 g/L. The amount of lipid recovery per g of algae was 0.31 g. The amount of lipid recovery per g of algae when an alkali treatment was performed was 0.27 g.

### Comparative Example 3

The process was performed in the same manner as in Comparative Example 2 except that the thermal treatment was omitted. The amount of lipid recovery per g of algae was 0.29 g. The amount of lipid recovery per g of algae when an alkali treatment was performed was 0.29 g.

### Example 2

### —Preculture—

As the microalgae belonging to Heterokontophyta, Nannochloropsis salina was dispersed in 10 L of a culture solution having a salt concentration of 36 g/L (3.6%). At the onset of the culture, the culture solution had an initial sodium nitrate concentration of 364 mg/L (60.0 mg/L as the initial nitrogen concentration), and the concentration of sodium dihydrogen phosphate dihydrate was 30 mg/L. The algal concentration after 14-day culture was 1.64 g/L.

### —Main Culture—

Two L of 10 L of the precultured solution (20% of the culture solution) was placed in a polycarbonate bottle. To the culture solution, 8 L of salt-added artificial seawater having a salt concentration of 36 g/L (3.6%) was added as a diluent (dilution ratio: 5 fold), and the main culture was started and continued for 7 days. The initial nitrogen concentration at the onset of the main culture was 0 mg/L, and the algal concentration at the end of the main culture was 0.63 g/L.

### —Thermal Treatment—

The pH of the culture solution after the main culture was adjusted to 5.0 with 1M HCl, and then the culture solution was maintained at 50° C. for 72 hours. The amount of lipid recovery per g of algae was 0.43 g. The amount of lipid recovery per g of algae when an alkali treatment was performed was 0.49 g.

### Comparative Example 4

After the main culture performed in the same manner as in Example 2, the pH of the culture solution was adjusted to 5.0, but no thermal treatment was performed. The amount of lipid recovery per g of algae was 0.10 g. The amount of lipid recovery per g of algae when an alkali treatment was performed was 0.49 g.

### Comparative Example 5

The process was performed in the same manner as in Comparative Example 4 except that the pH of the culture solution after the main culture was adjusted to 7.0. The amount of lipid recovery per g of algae was 0.10 g. The amount of lipid recovery per g of algae when an alkali treatment was performed was 0.49 g.

Table 3 collectively shows the conditions and results of Examples 1 and 2 and Comparative Examples 1 to 5. In a situation where no environmental stress was applied, no difference was found between the amount of lipid recovery from the sample not treated with alkali and that from the sample treated with alkali, even if no thermal treatment was performed. The amount of lipid recovery was not reduced through the thermal treatment. On the other hand, in a situation where the environmental stress was applied, performing the alkali treatment only without the thermal treatment increased the amount of lipid recovery as compared with the case where no environmental stress was applied. However, if neither the thermal treatment nor the alkali treatment was performed, the amount of lipid recovery was reduced as compared with the case where no environmental stress was applied. If the sample to which the environmental stress had been applied was thermally treated, the amount of lipid recovery was as large as the case where the sample was treated with alkali. Even if conditions of environmental stress were different, the same tendency was shown.

TABLE 3

| | | | Com. | Com. | Com. | | Com. | Com. |
|---|---|---|---|---|---|---|---|---|
| | | Ex. 1 | Ex. 1 | Ex. 2 | Ex. 3 | Ex. 2 | Ex. 4 | Ex. 5 |
| Preculture | Salt concentration (g/L) | 36 | ← | ← | ← | 36 | ← | ← |
| | Initial nitrogen | 60 | ← | ← | ← | 60 | ← | ← |
| | concentration (mg/L) | | | | | | | |
| | Culture time (day) | 10 | ← | ← | ← | 14 | ← | ← |
| | Culture temperature (° C.) | 25 | ← | ← | ← | 25 | ← | ← |
| | Final algal | 0.9 | ← | ← | ← | 1.64 | ← | ← |
| | concentration (g/L) | | | | | | | |
| Main | Salt concentration (g/L) | 36 | ← | ← | ← | 36 | ← | ← |
| culture | Initial nitrogen | 0.9 | ← | 54.9 | ← | 0 | ← | ← |
| | concentration (mg/L) | | | | | | | |
| | Culture time (day) | 24 | ← | 25 | ← | 7 | ← | ← |
| | Culture temperature (° C.) | 25 | ← | ← | ← | 25 | ← | ← |
| Thermal | Temperature (° C.) | 50 | — | 50 | — | 50 | — | — |
| treatment | pH | 5 | — | 5 | — | 5 | ← | 7 |
| | Treatment time (hr) | 72 | — | 72 | — | 72 | — | — |

| | | | | | | | |
|---|---|---|---|---|---|---|---|
| Amount of lipid recovered (g) | 0.53 | 0.26 | 0.31 | 0.29 | 0.43 | 0.10 | 0.10 |
| Amount of lipid recovered after | 0.53 | 0.52 | 0.27 | 0.29 | 0.49 | 0.49 | 0.49 |
| alkaline treatment (g) | | | | | | | |

### Example 3

The process was performed in the same manner as in Example 2 except that the thermal treatment was performed for five hours. The amount of lipid recovery per g of algae was 0.27 g. The amount of lipid recovery per g of algae when an alkali treatment was performed was 0.54 g.

### Example 4

The process was performed in the same manner as in Example 2 except that the thermal treatment was performed for 10 hours. The amount of lipid recovery per g of algae was 0.34 g. The amount of lipid recovery per g of algae when an alkali treatment was performed was 0.52 g.

### Example 5

The process was performed in the same manner as in Example 2 except that the thermal treatment was performed for 24 hours. The amount of lipid recovery per g of algae was 0.33 g. The amount of lipid recovery per g of algae when an alkali treatment was performed was 0.52 g.

### Example 6

The process was performed in the same manner as in Example 2 except that the thermal treatment was performed for 48 hours. The amount of lipid recovery per g of algae was 0.39 g. The amount of lipid recovery per g of algae when an alkali treatment was performed was 0.48 g.

Table 4 collectively shows the conditions and results of Examples 2 to 6. Performing the thermal treatment increased the amount of lipid recovery as compared with that of Comparative Example 4 where no thermal treatment was performed. The amount of lipid recovery had a tendency to increase with the increase in treatment time.

TABLE 4

| | | Ex. 2 | Ex. 3 | Ex. 4 | Ex. 5 | Ex. 6 |
|---|---|---|---|---|---|---|
| Preculture | Salt concentration (g/L) | 36 | ← | ← | ← | ← |
| | Initial nitrogen | 60 | ← | ← | ← | ← |
| | concentration (mg/L) | | | | | |
| | Culture time (day) | 14 | ← | ← | ← | ← |
| | Culture temperature (° C.) | 25 | ← | ← | ← | ← |
| | Final algal | 1.64 | ← | ← | ← | ← |
| | concentration (g/L) | | | | | |
| Main | Salt concentration (g/L) | 36 | ← | ← | ← | ← |
| culture | Initial nitrogen | 0 | ← | ← | ← | ← |
| | concentration (mg/L) | | | | | |
| | Culture time (day) | 7 | ← | ← | ← | ← |
| | Culture temperature (° C.) | 25 | ← | ← | ← | ← |
| Thermal | Temperature (° C.) | 50 | ← | ← | ← | ← |
| treatment | pH | 5 | ← | ← | ← | ← |
| | Treatment time (hr) | 72 | 5 | 10 | 24 | 48 |

| | | | | | |
|---|---|---|---|---|---|
| Amount of lipid recovered (g) | 0.43 | 0.27 | 0.34 | 0.33 | 0.39 |
| Amount of lipid recovered after | 0.49 | 0.54 | 0.52 | 0.52 | 0.48 |
| alkaline treatment (g) | | | | | |

### Example 7

The process was performed in the same manner as in Example 5 except that the temperature of the culture solution during the thermal treatment was set to 40° C. The amount of lipid recovery per g of algae was 0.31 g. The amount of lipid recovery per g of algae when an alkali treatment was performed was 0.52 g.

### Example 8

The process was performed in the same manner as in Example 5 except that the temperature of the culture solution during the thermal treatment was set to 65° C. The amount of lipid recovery per g of algae was 0.25 g. The amount of lipid recovery per g of algae when an alkali treatment was performed was 0.54 g.

### Example 9

The process was performed in the same manner as in Example 5 except that the temperature of the culture solution during the thermal treatment was set to 80° C. The amount of lipid recovery per g of algae was 0.18 g. The amount of lipid recovery per g of algae when an alkali treatment was performed was 0.51 g.

### Comparative Example 6

The process was performed in the same manner as in Example 5 except that the temperature of the culture solution during the thermal treatment was set to 25° C. The amount of lipid recovery per g of algae was 0.10 g. The amount of lipid recovery per g of algae when an alkali treatment was performed was 0.49 g.

Table 5 collectively shows the conditions and results of Examples 5 and 7 to 9, and Comparative Example 6. Performing the thermal treatment at a temperature higher than 25° C. increased the amount of lipid recovery as compared with the case where no thermal treatment was performed.

TABLE 5

| | | | | | | Com. |
|---|---|---|---|---|---|---|
| | | Ex. 5 | Ex. 7 | Ex. 8 | Ex. 9 | Ex. 6 |
| Preculture | Salt concentration | 36 | ← | ← | ← | ← |
| | (g/L) | | | | | |
| | Initial nitrogen | 60 | ← | ← | ← | ← |
| | concentration (mg/L) | | ← | ← | ← | ← |
| | Culture time (day) | 14 | ← | ← | ← | ← |
| | Culture temperature (° C.) | 25 | ← | ← | ← | ← |
| | Final algal | 1.64 | ← | ← | ← | ← |
| | concentration (g/L) | | | | | |
| Main | Salt concentration (g/L) | 36 | ← | ← | ← | ← |
| culture | Initial nitrogen | 0 | ← | ← | ← | ← |
| | concentration (mg/L) | | | | | |
| | Culture time (day) | 7 | ← | ← | ← | ← |
| | Culture temperature (° C.) | 25 | ← | ← | ← | ← |
| Thermal | Temperature (° C.) | 50 | 40 | 65 | 80 | 25 |
| treatment | pH | 5 | ← | ← | ← | ← |
| | Treatment time (hr) | 24 | ← | ← | ← | ← |

| | | | | | |
|---|---|---|---|---|---|
| Amount of lipid recovered (g) | 0.33 | 0.31 | 0.25 | 0.18 | 0.10 |
| Amount of lipid recovered after | 0.52 | 0.52 | 0.54 | 0.51 | 0.49 |
| alkaline treatment (g) | | | | | |

### Example 10

The process was performed in the same manner as in Example 5 except that the pH was set to 2.0. The amount of lipid recovery per g of algae was 0.20 g. The amount of lipid recovery per g of algae when an alkali treatment was performed was 0.46 g.

### Example 11

The process was performed in the same manner as in Example 5 except that the pH was set to 3.5. The amount of lipid recovery per g of algae was 0.36 g. The amount of lipid recovery per g of algae when an alkali treatment was performed was 0.58 g.

### Example 12

The process was performed in the same manner as in Example 5 except that the pH was set to 9.5. The amount of lipid recovery per g of algae was 0.18 g. The amount of lipid recovery per g of algae when an alkali treatment was performed was 0.58 g.

### Example 13

The process was performed in the same manner as in Example 5 except that the pH was set to 10.0. The amount of lipid recovery per g of algae was 0.19 g. The amount of lipid recovery per g of algae when an alkali treatment was performed was 0.56 g.

Table 6 collectively shows the conditions and results of Examples 5 and 10 to 13. Through the thermal treatment performed with the pH in the range of 2.0 to 10.0, the amount of lipid recovery was increased as compared with that of Comparative Examples 4 and 5 in which no thermal treatment was performed.

TABLE 6

| | | Ex. 5 | Ex. 10 | Ex. 11 | Ex. 12 | Ex. 13 |
|---|---|---|---|---|---|---|
| Preculture | Salt concentration (g/L) | 36 | ← | ← | ← | ← |
| | Initial nitrogen | 60 | ← | ← | ← | ← |
| | concentration (mg/L) | | | | | |
| | Culture time (day) | 14 | ← | ← | ← | ← |
| | Culture temperature (° C.) | 25 | ← | ← | ← | ← |
| | Final algal | 1.64 | ← | ← | ← | ← |
| | concentration (g/L) | | | | | |
| Main | Salt concentration (g/L) | 36 | ← | ← | ← | ← |
| culture | Initial nitrogen | 0 | ← | ← | ← | ← |
| | concentration (mg/L) | | | | | |
| | Culture time (day) | 7 | ← | ← | ← | ← |
| | Culture temperature (° C.) | 25 | ← | ← | ← | ← |
| Thermal | Temperature (° C.) | 50 | 40 | 65 | 80 | 25 |
| treatment | pH | 5 | 2 | 3.5 | 9.5 | 10 |
| | Treatment time (hr) | 24 | ← | ← | ← | ← |

| | | | | | |
|---|---|---|---|---|---|
| Amount of lipid recovered (g) | 0.33 | 0.20 | 0.36 | 0.18 | 0.19 |
| Amount of lipid recovered after | 0.52 | 0.46 | 0.58 | 0.58 | 0.56 |
| alkaline treatment (g) | | | | | |

### Example 14

### —Preculture—

As the microalgae belonging to Heterokontophyta, Nannochloropsis salina was dispersed in 10 L of a culture solution having a salt concentration of 36 g/L (3.6%). At the onset of the culture, the culture solution had an initial sodium nitrate concentration of 375 mg/L (61.8 mg/L as the initial nitrogen concentration), and the concentration of sodium dihydrogen phosphate dihydrate was 30 mg/L. The algal concentration after 8-day culture was 0.98 g/L.

### —Main Culture—

Two L of 10 L of the precultured solution (20% of the culture solution) was placed in a polycarbonate bottle. To the culture solution, 8 L of salt-added artificial seawater having a salt concentration of 36 g/L (3.6%) was added as a diluent (dilution ratio: 5 fold), and the main culture was started and continued for 7 days. At the onset of the main culture, the culture solution had an initial sodium nitrate concentration of 63 mg/L (10.3 mg/L as the initial nitrogen concentration), and the concentration of sodium dihydrogen phosphate dihydrate was 30 mg/L. The algal concentration at the end of the main culture was 0.66 g/L.

### —Thermal Treatment—

The pH of the culture solution after the main culture was adjusted to 5.0 with 1M HCl, and then the culture solution was maintained at 50° C. for 72 hours. The amount of lipid recovery per g of algae was 0.31 g. The amount of lipid recovery per g of algae through an alkali treatment was 0.32 g.

### Comparative Example 7

The process was performed in the same manner as in Example 14 was performed except that the thermal treatment was omitted. The algal concentration at the end of the main culture was 0.66 g/L, and the amount of lipid recovery per g of algae was 0.21 g. The amount of lipid recovery per g of algae when an alkali treatment was performed was 0.36 g.

Table 7 collectively shows the conditions and results of Example 14 and Comparative Example 7.

TABLE 7

| | | Ex. 14 | Com. Ex. 7 |
|---|---|---|---|
| Preculture | Salt concentration (g/L) | 36 | ← |
| | Initial nitrogen | 61.8 | ← |
| | concentration (mg/L) | | |
| | Culture time (day) | 8 | ← |
| | Culture temperature (° C.) | 25 | ← |
| | Final algal concentration (g/L) | 0.98 | ← |
| Main | Salt concentration (g/L) | 36 | ← |
| culture | Initial nitrogen | 10.3 | ← |
| | concentration (mg/L) | | |
| | Culture time (day) | 7 | ← |
| | Culture temperature (° C.) | 25 | ← |
| Thermal | Temperature (° C.) | 50 | — |
| treatment | pH | 5 | — |
| | Treatment time (hr) | 72 | — |

| | | |
|---|---|---|
| Amount of lipid recovered (g) | 0.31 | 0.21 |
| Amount of lipid recovered after alkaline | 0.32 | 0.36 |
| treatment (g) | | |

### Example 15

The lipid obtained in each of Examples can be used for the hydrogenation by the process [A]. This process can produce aliphatic alcohol and/or glycerin.

The lipid thus obtained can also be used for the transesterification by the process [B]. This process can produce aliphatic alkyl ester and/or glycerin. The aliphatic alkyl ester thus obtained can also be used for the hydrogenation by the process [C]. This process can produce aliphatic alcohol.

Further, the lipid thus obtained can also be used for the hydrolysis by the process [D]. This process can produce fatty acid and/or glycerin. The fatty acid thus obtained can also be used for the hydrogenation by the process [E]. This process can produce aliphatic alcohol.

### INDUSTRIAL APPLICABILITY

According to the algae treatment method of the present disclosure, even if the accumulation of lipid in the algae is increased through the application of an environmental stress, the recovery of lipid from the algae can be as easy as, or easier than, the recovery from the algae with no environmental stress. Thus, the algae treatment method of the present disclosure is useful for the treatment of the algae in the field of biomass technology.

1. An algae treatment method, comprising:
a first step of culturing microalgae belonging to Heterokontophyta in a culture medium having an initial nitrogen concentration of less than 12 mg/L for three days or more; and
a second step of performing a thermal treatment of the microalgae that have gone through the first step at a pH of 2.0 or more and 10.0 or less and a temperature of 35° C. or more and 80° C. or less.

2. The algae treatment method of claim 1, wherein
the second step is performed at a pH of 3.5 or more and 9.5 or less.

3. The algae treatment method of claim 1, wherein
the second step is performed at a temperature of 40° C. or more and 65° C. or less.

4. The algae treatment method of claim 1, wherein
the microalgae are of the genus Nannochloropsis.

5. The algae treatment method of claim 1, wherein
the thermal treatment is performed for five hours or more.

6. The algae treatment method of claim 1, wherein
the thermal treatment is performed for 48 hours or more.

7. The algae treatment method of claim 1, further comprising:
a culturing step of culturing the microalgae in a culture medium having an initial nitrogen concentration of 12 mg/L or more for three days or more, the culturing step being performed prior to the first step.

8. The algae treatment method of claim 7, wherein
the first step is performed using the culture solution that has gone through the culturing step and has been diluted 1.6-fold or more and 20-fold or less.

9. A method for producing lipid, the method comprising:
recovering the lipid from the algae that have been treated through the method of claim 1.

10. The method of claim 9, wherein
the lipid contains fats and oils.

11. A method of producing aliphatic alcohol and/or glycerin, the method comprising:
performing hydrogenation of the fats and oils produced through the method of claim 10 in the presence of a catalyst.

12. A method of producing aliphatic alkyl ester and/or glycerin, the method comprising:
performing transesterification of the fats and oils obtained through the method of claim 10 with lower alcohol having a carbon number of 1 to 5 in the presence of a catalyst.

13. A method of producing aliphatic alcohol, the method comprising:
performing hydrogenation of the aliphatic alkyl ester produced through the method of claim 12 in the presence of a catalyst.

14. A method of producing fatty acid and/or glycerin, the method comprising:
hydrolyzing the fats and oils obtained through the method of claim 10.

15. A method of producing aliphatic alcohol, the method comprising:
performing hydrogenation of the fatty acid produced through the method of claim 14.
